(19) United States
(12) Patent Application Publication
(10) Pub. No.: US 2017/0096713 A1
(43) Pub. Date: Apr. 6, 2017

(54) APPLICATIONS OF SINGLE MOLECULE SEQUENCING

(71) Applicants: California Institute of Technology, Pasadena, CA (US); Fluidigm Corporation, South San Francisco, CA (US)

(72) Inventors: Stanley N. Lapidus, Bedford, NH (US); Stephen R. Quake, Stanford, CA (US)

(21) Appl. No.: 15/266,787

(22) Filed: Sep. 15, 2016

Related U.S. Application Data
(63) Continuation of application No. 11/067,102, filed on Feb. 25, 2005, now abandoned.
(60) Provisional application No. 60/548,704, filed on Feb. 27, 2004.

(51) Int. Cl. C12Q 1/68

(52) U.S. Cl. CPC C12Q 1/6886; C12Q 1/6869; C12Q 2600/156

(57) ABSTRACT

The invention provides methods for determining the presence of a disease by comparing a sequence from a single target molecule with a predetermined sequence that is associated with a specific disease.

### CROSS-REFERENCES TO RELATED APPLICATIONS

This application is a continuation of U.S. patent application Ser. No. 11/067,102, filed on Feb. 25, 2005, which claims the benefit of U.S. Application No. 60/548,704, filed Feb. 27, 2004, the disclosures of which are incorporated by reference herein.

### STATEMENT AS TO RIGHTS TO INVENTIONS MADE UNDER FEDERALLY SPONSORED RESEARCH AND DEVELOPMENT

This invention was made with government support under Grant No. HG001642 awarded by the National Institutes of Health. The government has certain rights in the invention.

### REFERENCE TO A SEQUENCE LISTING

The Sequence Listing written in file SequenceListing 1023999.txt, created on Sep. 14, 2016, 690 bytes, machine format IBM-PC, MS-Windows operating system, is hereby incorporated by reference in its entirety for all purposes.

### TECHNICAL FIELD OF THE INVENTION

The invention relates to methods and devices for sequencing a nucleic acid, and more particularly, to practical applications of single molecule sequencing methods and devices

### BACKGROUND OF THE INVENTION

Bulk nucleic acid sequencing methods have resulted in widespread availability of several consensus genomic sequences, most notably that of humans. Bulk techniques, such as Sanger sequencing and others, rely on electrophoretic separation of nucleic acid fragments followed by piecing of the fragments together in order to obtain a representation of an entire target sequence. Those techniques result in a consensus sequence that may be representative of an entire group of organisms. However, they do not have the resolving power to provide specific genetic information about individual members of the group, or to detect changes that have epidemiologic, diagnostic, or therapeutic significance. For example, bulk sequencing methods do not typically reveal precise sequence information characteristic of an individual. Moreover, bulk sequencing is too slow and too expensive to be justifiable as a routine screening method. Such techniques are not ideal for analyzing disease-related differences across individuals. Finally, such techniques are not well-suited for detecting epidemiologic trends that provide insight into the spread of disease, the appearance of new diseases, or the susceptibility of individuals to disease.

Single molecule sequencing provides an opportunity to identify variations in nucleic acids at a resolution not feasible with bulk sequencing techniques. Also, unlike conventional sequencing methods, single molecule sequencing is not limited by the resolving power of electrophoretic separation. Thus, single molecule techniques have the potential to operate with increased sensitivity and longer read lengths, while providing more rapid and robust data as compared to conventional methods of sequencing.

The present invention provides applications for single molecule sequencing in the areas of diagnostics, therapeutics, research, and epidemiology.

### SUMMARY OF THE INVENTION

The invention provides methods for detection of genetic events with single molecule resolution. Methods of the invention are useful as applications of single molecule sequencing for disease detection, therapeutic intervention, epidemiologic analysis, cellular identification, gene expression analysis, developmental biology, immunology, and others. Single molecule sequencing offers the opportunity to elucidate genetic and biological characteristics of individual cells, to compare individual cells, and to obtain information that reveals genetic characteristics associated with biological function and dysfunction. Methods of the invention are not susceptible to the stochastic variance that is expected in bulk sequencing methods. The results of traditional amplification-based sequencing methods depend, in large part, on a random choice of templates that are amplified in the first few rounds. Primarily templates that are present in large numbers are amplified initially, subsequently making it difficult or impossible to detect a rare sequence event in a heterogeneous sample. Single molecule techniques facilitate determination of the sequences of a plurality of single-strands, rather than providing aggregate sequence that is representative of, for example, both copies of a target sequence in a cell population, or multiple cells types in a biopsy, or multiple organisms in a pooled sample.

Methods of the invention comprise determining the sequence of a singe nucleic acid template by synthesizing its complementary strand and imaging during each step of the polymerization reaction. In preferred embodiments, a primer nucleic acid is hybridized to a template and a polymerase is used to add sequential nucleotides to the complementary (primer) strand. The primer/template duplexes are adhered to a surface and spaced apart sufficiently such that at least a plurality of them are individually optically resolvable. Thus, resolution of the time between successive incorporations is all that is necessary to uniquely identify the linear sequence of the complementary strand, which in turn provides the template sequence. Methods of the invention may be carried out using single molecule fluorescence detection with conventional microscopes.

Essentially, single molecule sequencing according to the invention comprises exposing a surface-bound template nucleic acid to a nucleic acid primer, a polymerase, and labeled nucleotides. As individual nucleotides are added to the complementary strand, the label attached to the nucleotides is detected and the location of each incorporated nucleotide on the surface is recorded. The sequence of the template is assembled as nucleotides at each position along the complement are identified and recorded.

Preferably, methods of the invention are conducted in a parallel fashion in order to rapidly compile sequence data from a large number of templates on a single surface. Ideally, templates bound to a surface are individually optically resolvable from one another. Template nucleic acids are bound, directly or indirectly, to a surface for detection by any acceptable means, such as a chemical linkage or any other means capable of securing a template to a surface. In some embodiments, chemical linkages for attaching template nucleic acids comprise biotin/streptavidin, digoxigenin/anti-digoxigenin, or others known in the art. Likewise, the surface to which templates are attached may be any surface that presents acceptable attachment chemistries. Preferred surfaces are epoxides and polyelectrolyte multilayers. Preferred substrates include glass, quartz slides, silicon or commonly-available nucleic acid array chips. Other substrates useful in the invention are metal, nylon, gel matrix or composites. In some embodiments of the invention, the substrate is chemically modified to promote template attachment, improve spatial resolution, and/or reduce background. Exemplary substrate coatings include polyelectrolyte multilayers (PEM) and epoxides. Typically, a PEM is synthesized via alternate coatings with positive charge (e.g., polyllylamine) and negative charge (e.g., polyacrylic acid). Alternatively, a surface is covalently modified using, for example, vapor phase coatings using 3-aminopropyltrimethoxysilane.

Labeled nucleotides for use in the invention are any nucleotide that has been modified to include a label that is directly or indirectly detectable. In preferred methods, fluorescent labels are used to aid optical detection. The type of fluorescent label is selected based upon convenience and the detection device used. Cyanogen or dye molecules and other photolabile detection means may also be used. Preferred labels comprise fluorescent dyes, such as fluorescein, rhodamine, derivatized rhodamine dyes, such as TAMRA, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY,120 ALEXA, or a derivative or modification of any of the foregoing.

In one preferred embodiment, fluorescence resonance energy transfer (FRET) is used to generate an optical signal. In a single-pair FRET reaction, a donor fluorophore excites acceptor molecules within only a small radius, creating a high-resolution near-field radiation source that is superior to conventional near-field microscopy. Excitation of a fluorescent donor and emission by the acceptor occur at distinct wavelengths and a donor fluorophore is unlikely to excite distant surface debris, accordingly, background fluorescence is reduced. Various alternatives for detecting single nucleotides are disclosed in Braslavasky, et al., PNAS(USA) 100:7 3960-3964 (2003), the entirety of which is incorporated by reference herein.

Methods and compositions of the invention additionally contemplate conducting multiple sequencing by synthesis reactions on a single arrayed substrate. In one embodiment, sequencing by synthesis reactions are conducted on multiple templates derived from a plurality of sources, using a single solid support. Sequencing samples, comprising collections of nucleic acid templates, are deposited in uniquely-identifiable, self-contained locations. Each location may contain a plurality of nucleic acid binding sites that are individually optically resolvable for single molecule sequencing. The invention contemplates methods of depositing and arranging sequencing templates in order to assemble a complex collection of sequencing information. A collection of samples for use in the invention may be derived from a single patient, from many patients with the same or different health condition, or another random or non-random set of sources. The invention may be of particular relevance in simultaneously diagnosing illness in one or more patients, in determining responses to particular pharmaceuticals and therapeutics, or, additionally, in other medical or research applications.

Methods of the invention are useful to provide insight into disease progression, disease status, therapeutic effectiveness, and other parameters surrounding therapeutic intervention. For example, single molecule sequencing is useful to identify diseased cells (e.g. cancer cells or infected cells) in a tissue or body fluid sample obtained from a patient. Such methods are useful in diagnosis as well as therapy. Applying methods of the invention, a change in the number of diseased cells in response to therapeutic intervention is determined as an indicator of therapeutic effectiveness. Accordingly, methods of the invention comprise single molecule sequencing of cells obtained from a patient sample in order to assess the disease status of the patient from whom the sample is obtained. Methods of the invention are applicable to determine initial disease state as well as therapeutic progression, disease typing, and other aspects of therapeutic management. The results of applying methods of the invention also influence the choice of therapeutic intervention.

Methods of the invention are applicable to the identification of and subsequent intervention in diseases characterized by nucleic acid sequence mutations or variations. Cancer is a prominent mutation-associated disease, characterized by genomic changes that alter the ability of cells to control proliferation and growth. Methods of the invention provide rapid and sensitive sequence analysis, and thus, are especially useful in cancer detection, diagnosis and research. For example, methods of the invention have the ability to detect sequences present in only a small percentage of cells in a sample. This high level of specificity is beneficial in early detection of cancer in an individual patient, when the population of cancer or precancer cells is still comparatively small. In one embodiment, methods of the invention may be used for screening human tissue or other samples, such as blood, bone marrow, cervical scrapings, or stool. In the early stages of disease, only a few cells collected may have mutations indicative of cancer. By obtaining sequence information from individual DNA strands, rather than collective sequence information, methods of the invention allow identification of point mutations, small deletions, and other alterations in a small population of cells, based on genomes of individual cells. Obtained sequence information then is compared to information in a database of sequences known to be associated with a specific disease state. For example, sequence obtained from a patient sample may be compared to a database of sequences known to be associated with cancer or with some other disease (e.g., an infective agent). Matching algorithms are used to determine a match with a sequence in the database, thus aiding diagnosis. The same methods are useful for therapeutic choice. For example, methods of the invention are used to obtained sample sequence information from diseased cells that is then compared to sequence information from previous patients who have been successfully or unsuccessfully treated. Because therapeutic response is often based upon underlying genetics, comparison of relevant sequence from an individual to sequences associated with successful therapeutic treatment, aids in the selection of a therapy with an increased probability of successful treatment.

Methods of the invention provide further diagnostic-related applications in cancer, such as metastasis analysis and recurrence monitoring. Sequence information from lymph node samples and tumor margin cells provides more definitive diagnosis of tumor boundaries and tumor spread than pathology analysis alone. Furthermore, isolated groups of cells may be selected from a pathology slide, to serve as template sources for single molecule sequencing.

The invention also provides methods for using single molecule sequencing in order to guide therapeutic choice. Often, especially in cancer, whether a patient responds to a given therapy depends upon tumor genotype. In some embodiments, methods of the invention are useful in identifying altered genes implicated in tumor cell proliferation. Molecular characterization of tumors through knowledge of gene-specific mutations will facilitate informed decisions about choosing targeted therapies. Conversely, if it is determined, for example, that a patient's tumor harbors a mutation in a particular gene that is known to causes resistance to a specific chemotherapeutic agent, then an informed choice may be made among other available therapies. Thus, specific, accurate, and rapid knowledge of tumor sequences provides valuable information in selecting a therapeutic regimen.

Methods of the invention are also useful to identify amplifications or deletions in genomic DNA that are associated with disease. Traditional methods for the detection of genomic loss, such as PCR-based loss of heterozygosity analysis or Southern Blotting, require the use of large numbers of cells in order to generate sufficient genomic DNA to accurately detect a significant loss of chromosomal material. In contrast, single molecule sequencing provides digital information regarding the presence or absence of a critical amount of nucleic acid material. Thus, instead of large numbers of cells, one needs only a sufficient number of template strands to determine if a loss of genomic material has occurred. In one embodiment, methods of the invention comprise comparing genomic sequences from normal patient germ line cells to tumor cells of the patient, wherein any sequence differences are attributable to cancer or precancer. Furthermore, methods of the invention are useful to identify genomic amplifications, deletions, and rearrangements in gamete screening, pre-implantation screening, and prenatal testing.

Single molecule sequencing as described herein provides the ability to generate an essentially-complete catalog of genetic alterations associated with diseases or disease susceptibility. Such knowledge, in turn, leads to more effective diagnostic and therapeutic options, and is particularly advantageous with respect to cancer and other complex genetic diseases. Thus, in a preferred embodiment of the invention, high-speed single molecule sequencing is used to sequence DNA from a multiplicity of normal and diseased cells in order to generate a catalog of mutations, other alterations, and alleles suspected to be associated with disease. Additionally, methods of the invention facilitate retrospective analysis of tumors and diseased tissue because cellular samples are not limited to fresh tissue or fluid specimens. Due to the sensitivity of single molecule sequencing, specimens in paraffin blocks, specimens otherwise fixed on pathology slides, and other archival specimens may be used as sources of sequencing templates. Once generated, such a catalog is useful as a diagnostic tool as well as a tool to guide therapeutic decision making as, for example, in the choice of an effective chemotherapeutic agent.

Rapid single molecule sequencing is also useful in the contexts of drug discovery and drug development. In a clinical drug trial, for example, methods of the invention are useful to analyze hypotheses about the genetic bases of positive responses or certain side effects to a particular drug. In one embodiment, the invention provides a rapid method to sequence the genomes or portions of the genomes of all subjects in a research study. Common polymorphisms or mutations in individuals who experienced the same side effect may be identified, providing valuable information about which patients should not be prescribed that drug in the future. Similar embodiments of the invention provide a rapid method of determining genetic profiles of persons who are likely to have a positive response to a particular drug. In another embodiment useful in drug development, the invention provides a method for identifying and measuring all transcripts in a cell that has been exposed to a particular drug, compared to an unexposed cell, to understand the effect that drug has on regulation of certain genes. Further elucidation is provided by correlating sequence with prior clinical outcome in other cases and/or with disease phenotype.

Methods of the invention are also useful in gene expression analysis. For example, in one embodiment, methods of the invention are used to generate an immune fingerprint. Single molecule sequencing of T-cell and/or B-cell expression provides insight into the immune repertoire of the subject from whom a sample is taken. Knowledge of immune cell expression patterns provides insight into not only the function of an individual's immune system, but also provides insight on a patient's response to therapeutic intervention, disease progression, and treatment options. Thus, in a preferred embodiment, the invention provides methods for determining and evaluating immune function, either on a cell-by-cell basis or on a population of immune cells by sequencing nucleic acids obtained from relevant immune cells.

Methods of the invention are also useful to monitor gene expression in other contexts.

For example, in one embodiment, gene expression in individual cells is tracked in order to gain insight into which cells in a population are true progenitor cells. Currently, there are few true progenitor cell markers, and it is often difficult to distinguish and isolate real progenitors. Single molecule sequencing, especially on a cell-by-cell basis, provides a set of molecular markers useful to uniquely identify progenitor cells, which then are easily isolated. Methods of the invention allow the rapid identification and isolation of progenitors. Thus, according to the invention, progenitor cells are identified by single molecule gene expression sequencing as taught herein.

Methods of the invention are also useful in epidemiology. Single molecule sequencing provides robust data useful for identifying and tracking disease. For example, in an infectious disease epidemiology application, tissue or body fluid samples are obtained from patients presenting with an illness, suspected to be caused by an infectious agent. Nucleic acids in the samples are sequenced and relevant sequence data are cataloged and stored. The sequence data are correlated with known diseases in order to allow rapid diagnosis and to allow epidemiologic tracking of disease outbreaks. Single molecule sequencing data also allows the rapid identification of new infectious diseases. Single molecule sequencing as described herein is able to identify the outbreak of a new disease. For example, the invention taught herein rapidly identifies a new disease, such as SARS, upon first presentation because the nucleic acid sequence of the newly isolated pathogen would not be in the database of disease sequences. The ability to rapidly identify new pathogens has an important impact on managing emerging infectious disease outbreaks. Single molecule sequencing provides for ubiquitous epidemiology as opposed to disease-specific epidemiology. Methods of the invention allow one to map an entire nucleic acid ecosystem in a patient sample which leads to the ability to match the patient's nucleic acid profile against essentially all known diseases or to identify a new disease at the first sign of outbreak.

### DETAILED DESCRIPTION OF THE INVENTION

The present invention relates to applications of single molecule sequencing. In particular, the invention relates to the recognition of nucleic acid events that are relevant to disease detection, monitoring, diagnosis, therapy, and management. Single molecule sequencing is a powerful tool capable of elucidating sequence-specific information on a single nucleic acid template. The ability to conduct single template sequencing allows the identification of subtle, often rare event, changes in nucleic acids that are important as the underlying basis for diseases such as cancer and others. Moreover, single molecule sequencing is an effective tool for epidemiology, developmental biology, and cell sorting and identification.

Single molecule sequencing provides the ability to analyze single nucleic acid templates in parallel and with a high degree of precision. Using an isolated nucleic acid sequence as the substrate, individual labeled nucleotides are added sequentially by a polymerase to a growing complement strand. A label is detected as each nucleotide is added to the strand and the template sequence is determined. Precise single molecule sequence determination as described in more detail below opens the door to numerous applications in biology and medicine, some of which are described below and others of which are apparent to the skilled artisan upon consideration of the present invention.

### Single Molecule Sequencing

Single molecule sequencing may take many forms. In one embodiment, the invention comprises exposing a nucleic acid primer to a template sequence in the presence of a polymerase and at least one labeled nucleotide base that is capable of hybridizing with a template nucleic acid downstream of the hybridized primer. Nucleotide bases may be selected from the common Watson-Crick bases, adenine, thymine, cytosine, guanine, and uracil, or may be modifications of those bases, such as peptide nucleic acids, ribonucleotides, or nucleotides modified to incorporate a detectable label (e.g., with linkers or adapters). As each nucleotide is added to the growing complement strand, its label is detected and its position on the template is noted. Once a sufficient number of nucleotides have been incorporated, a sequence is determined. Methods of the invention facilitate rapid whole genome sequencing. Methods of the invention, however, also contemplate partial genome sequencing to obtain template or fingerprint sequences, thereby facilitating even more rapid sequence comparisons. What follows is one example of a manner in which single molecule sequencing is conducted. Variants of the method described below are apparent to the skilled artisan.

### EXAMPLE 1

In this example, the sequence of a template DNA molecule was determined using an exemplary single molecule sequencing method. The sequencing substrate for immobilizing a target nucleic acid comprised a PEM surface. A fused silica microscope slide (1 mm thick, 25×75 mm size, Esco Cat. R130110) was used as the substrate for attachment of DNA templates.

The slides were first cleaned as follows. Slides were sonicated for 30 minutes in a solution of 2% Micro-90 in MilliQ water (20 mL Micro-90 in 980 mL water). The slides were then removed from the sonicator and rinsed under a cascading stream of MilliQ water. The slides were then placed into a fresh RCA solution (6:4:1 MilliQ H 20/NH 40H(28%)/H 2O 2 (30%)) and boiled at 60 C for 45 minutes. The slides were then rinsed in a stream of MilliQ H 2O, cooled to room temperature, and stored in MilliQ H 20.

A polyelectrolyte multilayer was produced on the RCA-cleaned slides described above. Prior to deposition of the PEM, separate solutions of polyethleneimine (PEI) and polyacrylic acid (PAA) were prepared. Separate solutions of PEI and PAA (2 gm/L each) were made by dissolving in MilliQ water. The pH was adjusted to 6.6 using dilute HCl. The resulting PAA solution was filtered through a 0.22 u filter flask, and the PEI solution was filtered through a 0.45 u filter. Two crystallizing dishes were filled (500 mL) with either PEI or PAA. The RCA-cleaned slides were then immersed first in the PEI solution for 10 minutes, followed by immersion in MilliQ water and thorough rinsing with cascading MilliQ water for 5 minutes. The slides were then immersed in PAA for 10 minutes, removed and rinsed with cascading MilliQ water. The cycle (PEI/rinse/PAA/rinse) was repeated 4 times. After the last cycle, the slides were placed in MilliQ water for storage.

The PEM-coated slides described above next were biotinylated. A 5 mL solution of 1-[3-(dimethylamino)propyl]-3-ethylcarbodiimide hydrochloride (EDC, 50 mM in 2-[N-morpholino]ethanesulfonic acid (MES) buffer)) was combined with 5 mL biotin solution in dilute MES to a total volume of 96 mL (2.5 mM EDC/Biotin in 86 mL MES buffer). The PEM slides were immersed in this solution with gentle agitation for 10 seconds and then rinsed in MES. This process was repeated 4 times in 100 mL volumes of EDC/Biotin to produce biotinylated PEM slides.

The biotinylated PEMs were then streptavidinated in preparation for duplex binding. Streptavdin-Plus (SA20, Prozyme) was dissolved in 10 mM Tris/10 mM NaCl buffer at 0.14 mg/ml (2.33 uM), and filtered with a 0.2 u filter. The biotinylated PEM slides were immersed in the streptavidin solution in a 100 mL beaker and incubated with stirring for 15 minutes. The slides were then removed and rinsed in 100 mL of 10 mM Tris-NaCl buffer with gentle agitation for 10 seconds. The slides were then rinsed in 5 clean volumes of 3×SSC-0.1% Triton. The slides were incubated for 10 minutes in the final rinse. The resulting streptavidinated slides were stored in 10 mM NaCl at 4 C.

Duplex (1 mM) comprising template having the sequence 5′-GTCGACTCCGATAAAGGATAAGTGCATAAGGGG-peg-Biotin (SEQ ID NO: 1) and a DXS17 primer with a 3′ cyanine-5 dye and 5′ cyanine-3 dye attached, Cy5-ATTTCCTATTCACGTATTCCCC-Cy-3 (SEQ ID NO: 2) in 10 mM MgSO 4, 10 mM (NH 4) 2SO4, 10 mM KCl, 0.1% Triton, 20 mM Tris (pH 8.8) was added to the streptavidinated slides prepared above. The Cy3 dye acted as a fluorescence resonance energy transfer (FRET) donor, and the Cy5 dye acted as the FRET acceptor. Duplex was imaged on the PEM surface after washing using an inverted TE2000-U microscope (Nikon) with a CFI-60 total internal reflection objective (1.45 NA, Nikon). The surface was exposed to light at 532 nm to excite the donor, and emission from the acceptor was observed at 635 nm to locate duplex on the surface. Next, the sample was bleached, and 1 μM of dGTP-Cy5 was added in the presence of 50 u/ml Klenow exo-polymerase in the above-described buffer (10 mM MgSO 4, 10 mM (NH 4) 2SO4, 10 mM KCl, 0.1% Triton, 20 mM Tris (pH 8.8)). After washing, fluorescence emission from the Cy5 acceptor was observed in order to determine which template molecules incorporated the dGTP. Photobleaching was then used to extinguish incorporated label, and the next labeled base was added for incorporation. The result of this process produced a series of images that, when stacked, produced the sequence of incorporations at each duplex location on the surface. The sequence of the template was confirmed based upon analysis of these images.

### Genomic DNA Analysis

High-speed single molecule detection allows patient-specific, as well as general, population-based knowledge concerning the genetic basis of diseases and disorders. Cancer is an example of a disease or disorder that has a strong genetic basis. Complete sequencing of large numbers of tumors using single molecule sequencing provides a catalog of somatic cell mutations (including, without limitation, deletions, additions, amplifications, rearrangements, substitutions, losses, translocations, methylation, and other alterations of genomic DNA) that is useful to diagnose, evaluate, prognosis, and treat patients. A catalog of disease-related mutations and other alterations is a powerful diagnostic tool useful to rapidly categorize samples sequenced from future patients. Moreover, single molecule sequencing allows one to identify previously-unknown mutations that may be associated with cancer. Finally, single molecule sequencing on pooled samples allows rapid identification of deletions, amplifications, and other changes that are indicative of cancer—even if the specific mutational change is not known.

Analysis of genomic DNA using single molecule sequencing provides an approach that allows rapid identification of a genomic change present in a sample in low amounts. The ability to quickly and accurately perform rare-event detection is of great significance for the early diagnosis of cancer. Many cancers, if detected early, are treatable, and if detected too late may not be treatable. Cancer begins as somatic cell mutations accumulate in a very small initial population of cells. In samples typically obtained for genomic analysis, cancer or precancer cells are in very low abundance compared to healthy somatic cells. Bulk mutation detection mechanisms typically fail to detect these rare event changes. A digital technique, such as single molecule sequencing, allows the sequencing through mutations in multiple single templates rapidly. This, in turn, allows the detection of the rare-event mutations underlying cancer or precancer.

In one embodiment of the invention, tumor DNA is obtained and prepared using standard methods. Approximately 10 times coverage of each genomic region is sequenced. Using single molecule sequencing, the genome of the cancer tissue is rapidly sequenced. Mutations, insertions, deletions, rearrangements, and other alterations present in the tumor DNA are detected. Sequence assembly is accomplished using standard alignment techniques, such as BLAST (www.ncibi.nlm.nih.gov), incorporated by reference herein. Tumor sequences are compared to known sequences for either normal or cancer tissue or to consensus sequences in order to identify changes associated with cancer. Newly discovered genomic changes (i.e., those not previously associated with cancer) are cataloged and become known to be associated with a particular disease over time. Thus, patients are rapidly and accurately diagnosed based upon their individual genomic complement, either before or at the time of symptomatic-presentation of a disease.

In another embodiment of the invention, DNA is isolated from a patient's tumor or other diseased sample and is compared to normal DNA from the same patient. Whole genome sequencing of both the tumor and normal DNA may be done rapidly on a parallel basis using single molecule sequencing as described above. Alternatively, only portions of the genome are sequenced and compared. Genome portions of interest include, for example, sequences associated with a known or candidate tumor suppressor gene or oncogene, or intronic sequences containing repeats that are susceptible to amplification by defective cellular machinery. Following sequence determination, a comparison is made between tumor and normal sequence. Differences between the tumor and normal sequences are identified as tumor-related mutations. In effect, any difference between the two likely is indicative of disease because all somatic cells should have the same sequence. Detection of a variation from the normal somatic cell sequence, indicating that a population of cells containing abnormal sequences is present, results in a positive diagnosis. Alternatively, patient tumor sequence may be compared to a normal banked or consensus sequence instead of the patient's own normal DNA.

In a related embodiment broad-based disease susceptibility testing is performed using single molecule sequencing on pooled genomic samples. For example, in a large population, the number of positive samples (i.e., those with a mutation present) is relatively small. Bulk sequencing likely would not detect mutations in pooled samples. Using high-resolution single molecule sequencing, however, any positive sample is detected with digital precision. Thus, according to the invention, genomic samples from a predetermined number of patients (the number of patients does not matter for purposes of the invention) are collected, pooled and sequenced using single molecule sequencing techniques as described above. Single molecule sequencing is done through large tracts of the genome, and mutations derived from any source are detected in the pooled sample. To determine the source of a mutation or mutations, the original collection of individual patient samples is divided in half, re-pooled, and resequenced. This process continues until a unique identification of the affected patient or patients is possible.

Due to the rapidity of single molecule sequencing, it is possible to perform multiple sequencing steps in a matter of hours or days. Using single molecule sequencing, pooled sequences, when compared to a consensus sequence, readily identify losses or amplifications in genomic DNA. All somatic cells will have not only the same sequence but will also be present in the same amounts. Deviations are detected using single molecule sequencing with fewer cells than in bulk sequencing because individual DNA molecules are sequenced instead of an amalgam of cells that typically provide the basis for bulk sequencing assays as, for example, in assays for loss of heterozygosity. In a related embodiment, data from a pooled experiment is useful for determining the frequency and distribution of mutations in a given population, without identifying the owners of specific mutations.

The rapid results provided by single molecule sequencing also allow sequencing to detect familial mutations. For example, if it is determined that a patient has a mutation indicative of a cancer, certain forms of which have a strong familial link (e.g., breast cancer, colon cancer), primary siblings typically are not tested unless specified criteria are met. Single molecule sequencing not only identifies the underlying mutation in the primary patient, but allows rapid, cost-effective sequencing of relatives who also might carry the mutation.

Single molecule sequencing is also useful to perform tumor typing. Tumor typing may involve determining a genetic profile for a particular patient's tumor in order to guide treatment or other decisions. For example, the standard treatment for patients with colon cancer is the drug 5-Fluorouracil (5FU). Although 5FU works to reduce tumors in many colon cancer patients, it actually accelerates tumor growth in a class of patients who have Hereditary Non-Polyposis Colorectal Cancer (HNPCC). HNPCC is a familial form of colon cancer with a distinct genetic profile that is ascertainable by sequencing cellular DNA. Thus, to avoid tumor acceleration in potential HNPCC patients, it is particularly important to know a colon cancer patient's genetic profile in order to determine the most effective treatment for that patient. Single molecule sequencing is useful to make that determination because it is rapid, reliable, and effectively digital, therefore promptly indicates the presence or absence of the relevant genetic event(s). Methods of the invention make possible the rapid and accurate identification of tumor-related mutations, thus an appropriate treatment may be selected or an inappropriate treatment avoided.

### Expression Analysis

Single molecule sequencing is also useful in gene expression analysis. Alteration in expression constructs is often indicative of a change in physiological status. Changes in expression patterns reflect cellular activities as well as disease state. Expression sequence analysis provides insight into the specialized activities of cells from different organs or of different types. Thus, expression analysis reveals aspects of the immune repertoire that are not apparent on a gross level. According to an aspect of the invention, a sequence determination is made with respect to a population of expressed B-cells. Single molecule sequencing offers rapid, high-throughput sequencing that reveals specific detail as to which immune cells are active, and the likely epitopes against which they function. Single molecule sequencing also provides an immune fingerprint that is used to identify an infection based upon the specifics of a patient's immune response. The immune fingerprint generated using single molecule sequencing is compared to a database of collected immune sequence data in order to identify an infection. New infections are tracked through the appearance of new sequence specificities either alone or in combination with other diagnostic techniques. Isolation of immune cells is well-known in the art, and application of the present invention to sequencing a patient's immune cell complement is contemplated by the present invention.

Single molecule sequencing also presents opportunities in the area of developmental biology. Sequence cues throughout development are indicative of critical biological and developmental activities. Because single molecule sequencing is useful to detect low-frequency nucleic acid sequences, it is used to detect fetal cells in maternal serum. Thus, fetal DNA and RNA are screened for inherited, as well as infectious, diseases via the maternal serum. This reduces complications often associated with amniocentesis. Single molecule sequencing is, however, useful to determine sequences from amniotic samples when amniocentesis is the preferred mode of sample production.

Single molecule sequencing is also useful in epidemiology. In a preferred embodiment, an appropriate patient sample is obtained and DNA in the sample is sequenced. Optionally, the patient's genomic DNA is excluded. A catalog is compiled comprising a fingerprint of the DNA (or RNA in other preferred embodiments) present in samples obtained from a multiplicity of patients. Each patient's disease status then is correlated with specific sequence information obtained from the patient's sample. In this way, diagnostic accuracy and verifiability is improved, as a patient's disease status is confirmed by comparing the patient's DNA to sequences in the database. As mentioned above, whole genome sequencing is optional. In some circumstances, it is necessary only to sequence sufficient nucleic acid to establish a fingerprint for comparison with future samples.

Ubiquitous epidemiology in which patient DNA is routinely sequenced and stored for disease identification and comparison with future samples is also useful to identify and track new disease outbreaks. For example, a patient who presents with a new DNA profile (i.e., containing a sequence that is not in the database) may be diagnosed with a new condition. Future patients presenting with the same nucleic acid profile are tracked. In this way, potential epidemic outbreaks are controlled. With respect to new diseases, no a priori assumptions are necessary. A novel sequence will immediately be identified as such, and appropriate monitoring can be put in place.

What is claimed is:

1. A method for detecting low abundance nucleic acids indicative of a disease state in a heterogeneous sample, the method comprising the steps of:
a) obtaining a biological sample suspected to contain a nucleic acid that would not be expected to be present in the sample if the individual from whom it was obtained were healthy;
b) conducting a sequencing reaction on nucleic acid in said sample; and
c) comparing nucleic acid sequences obtained in said conducting step to one or more reference sequences that represent nucleic acids that are not expected to be present in a sample obtained from a healthy individual, thereby to identify nucleic acids in said sample that are indicative of a disease state.

2. The method of claim 1, wherein said biological sample is blood or another body fluid.

3. The method of claim 1, wherein said biological sample is obtained from tissue.

4. The method of claim 1, wherein said reference sequences represent a mutation that is indicative of cancer or precancer.

5. The method of claim 1, wherein said reference sequences represent an infectious disease agent.

6. The method of claim 1, wherein said heterogeneous sample comprises nucleic acid derived from multiple cell types.

7. The method of claim 4, wherein said mutation is a mutation or a deletion.

8. The method of claim 1, wherein said biological sample is maternal blood.

9. The method of claim 8, wherein said reference nucleic acid is fetal DNA or RNA.

10. The method of claim 1, wherein said comparing step identifies the presence of nucleic acids derived from multiple organisms in a pooled sample.

11. A method for detecting a nucleic acid sequence in a heterogeneous sample, wherein said sample is suspected to contain a nucleic acid template that would not be expected to be present in said sample, the method comprising the steps of:
a) obtaining a heterogeneous sample, comprising a nucleic acid;
b) depositing said sample onto a substrate;
c) conducting a template dependent primer extension reaction on said sample, thereby obtaining sequence information for said heterogeneous sample; and
d) comparing a sequence obtained in said conducting step to a reference sequence, thereby detecting said nucleic acid template that would not be expected to be present in said sample.

12. The method of claim 11, wherein the sample is deposited onto the substrate such that at least a portion of nucleic acids contained in said sample are individually optically resolvable on said substrate.


SEQUENCE LISTING (2 sequences)

SEQ ID NO: 1 - DNA, 33 bp, Artificial Sequence
  Synthetic oligonucleotide
gtcgactccgataaaggataagtgcataagggg

SEQ ID NO: 2 - DNA, 22 bp, Artificial Sequence
  Synthetic oligonucleotide
atttcctattcacgtattcccc